(19) United States
(12) Patent Application Publication
(10) Pub. No.: US 2019/0046407 A1
(43) Pub. Date: Feb. 14, 2019

(54) SPIKE PORT FOR MEDICAL SOLUTION BAG ASSEMBLY AND RELATED METHODS

(71) Applicant: Fresenius Medical Care Holdings, Inc., Waltham, MA (US)

(72) Inventors: Kahina Rabia, Ogden, UT (US); Makenzie Jones, North Logan, UT (US); Christopher Yim Chau, Mission, TX (US); Eduardo Ignacio Muñoz Laverde, Bogota (CO)

(21) Appl. No.: 15/673,269

(22) Filed: Aug. 9, 2017

(51) Int. Cl. A61J 1/20; A61M 39/22; A61J 1/14; A61M 5/00; A61J 1/10; A61M 1/28; A61M 5/14

(52) U.S. Cl. CPC A61J 1/2089; A61M 39/22; A61J 1/1475; A61M 5/1411; A61J 1/10; A61M 1/28; A61M 5/002

(57) ABSTRACT

A molded spike port including a first portion defining a first chamber, a plug disposed in the first chamber and configured to form a seal with the first portion, a second portion defining a second chamber, the second portion including a sealing ring configured to seal the spike port to a spike, a tip configured to be inserted into a fluid bag, the tip comprising a membrane, and a wall between the first portion and the second portion, wherein the wall is configured to break when the first portion is twisted relative to the second portion.

### TECHNICAL FIELD

This specification relates generally to spike ports for medical solution bag assemblies and related methods.

### BACKGROUND

A spike port is often times connected to a medical solution bag as part of the bag closure. To use the medical solution in a treatment, a clinician typically removes a cover from the spike port and then inserts a spike into the spike port to break a barrier and provide access to the medical solution within the bag.

### SUMMARY

In one aspect, a molded spike port includes a first portion defining a first chamber and a plug disposed in the first chamber. The plug is configured to form a seal with the first portion. The molded spike port also includes a second portion defining a second chamber, the second portion including a sealing ring configured to seal the spike port to a spike. The molded spike port also includes a tip configured to be inserted into a fluid bag, the tip including a membrane and a wall between the first portion and the second portion. The wall is configured to break when the first portion is twisted relative to the second portion.

Implementations can include one or more of the following features.

In some implementations, the membrane has a thickness of 0.3 mm to 0.5 mm.

In some implementations, the sealing ring is configured to seal spikes of multiple configurations.

In some implementations one of the configurations is ISO-compliant and another of the configurations is non-ISO compliant.

In some implementations, the sealing ring has an inner diameter of 4.8 mm to 5.0 mm.

In some implementations, the tip of the spike port includes a smooth outer surface configured to seal the tip of the spike port to the fluid bag via heat sterilization.

In some implementations, the smooth outer surface has a surface roughness of 0.56 μm.

In some implementations, the plug is interference fit with the first chamber.

In some implementations, the wall comprises a thinned portion with a wall thickness that is thinner than a remainder of the wall.

In some implementations, the spike port is made from plasticized polyvinyl chloride.

In another aspect, a method of manufacturing a molded spike port includes injection molding a spike port. The method includes flowing a material through two injection mold gates on opposing sides of a tip zone of a spike port mold, the injection mold gates being positioned a predetermined length from an end of the tip of the mold. The method also includes filling a membrane zone of a mold such that material from the two injection mold gates meets in the membrane zone. The method also includes filling a tip zone of a spike port mold with material and compressing the material in the membrane zone to form a membrane with a predetermined membrane thickness.

Implementations can include one or more of the following features.

In some implementations, the predetermined membrane thickness is 0.3 mm to 0.5 mm.

In some implementations, the injection mold gates are positioned approximately 8.3 mm to 8.7 mm from the end of the tip zone of the mold.

In some implementations, a knit line is formed in the membrane zone when the material from the two injection mold gates meets in the membrane zone.

In some implementations, filling the tip zone of a spike port mold creates a spike port tip with a smooth outer surface.

In some implementations, the smooth outer surface has a surface roughness of 0.56 μm.

In some implementations, the method includes filling a first portion zone of a spike port mold and a second portion zone of a spike port mold such that the first portion and the second portion are connected at a thinned portion.

In some implementations, the method includes filling a first portion zone of a spike port mold and a second portion zone of a spike port mold such that the second portion and the tip are connected at a distance further from the end of the tip zone of the mold than the two injection mold gates.

In some implementations, a hydraulic cylinder compresses the material in the membrane zone to the predetermined thickness.

Implementations can include one or more of the following advantages.

In certain implementations, the molded spike port is manufactured using a two-step injection molding process that avoids producing a knit line on the exterior of the tip of the second portion of the molded spike port or results in a knit line of negligible size. A knit line is produced during manufacturing where at least two flow fronts meet and the material in the flow fronts does not meld, or “knit” together, completely when meeting. Because there is no knit line or a knit line of a negligible size, the molded spike port can be attached to a saline bag without the use of a solvent. The absence of a knit line on the exterior surface also decreases the likelihood of contamination of the saline solution inside the bag. Additionally, the manufacturing process repeatably creates uniform molded spike ports, reducing failures in attaching the molded spike ports to saline bags and in inserting spikes into the molded spike ports.

In certain implementations, the molded spike port is flexible and able to be used with ISO-compliant and non-ISO-compliant spikes.

In certain implementations, the molded spike port includes a plug disposed in the first portion of the spike port. The plug provides structural support to the first portion of the spike port which is removable. The structural support in the first portion of the spike port minimizes deformation of the channel in the spike port where the spike is inserted.

In certain implementations, the plug is positioned within the first portion of the spike port with an interference fit to provide a seal. Therefore, the plug does not need to be welded in place in the first portion of the spike port, which is advantageous because welding can cause warping of the spike port.

Other aspects, features, and advantages will be apparent from the description and drawings, and from the claims.

### BRIEF DESCRIPTION OF THE DRAWINGS

FIG. 1 is a schematic of a hemodialysis system configured for a priming procedure during which a saline bag assembly with a spike port is connected to a blood line set, which is operatively connected to a hemodialysis machine.

FIGS. 2A and 2B are schematics of the saline bag assembly of FIG. 1 with the spike port closure in a closed state before connection of the blood line set and in an open state after the connection of the blood line set, respectively.

FIGS. 3A, 3 B, and 3C are front, side, and top views, respectively, of the spike port of the saline bag assembly of FIGS. 2A and 2B.

FIG. 4 is an enlarged view of region 4 of a wall of the molded spike port in FIGS. 3A-3C.

FIGS. 5A-5B schematically represent a manufacturing process for making the molded spike port of FIGS. 3A-3C.

### DETAILED DESCRIPTION

During hemodialysis (“HD”), a patient's blood and a fluid (e.g., a dialysis solution or dialysate) pass through a dialyzer of a dialysis machine. A semi-permeable membrane in the dialyzer separates the blood from the dialysate within the dialyzer and allows diffusion and osmosis exchanges between the dialysate and the blood stream. These exchanges across the membrane remove waste products, including solutes like urea and creatinine, from the blood. These exchanges also regulate the levels of other substances, such as sodium and water, in the blood. In this way, the dialysis machine acts as an artificial kidney for cleansing the blood.

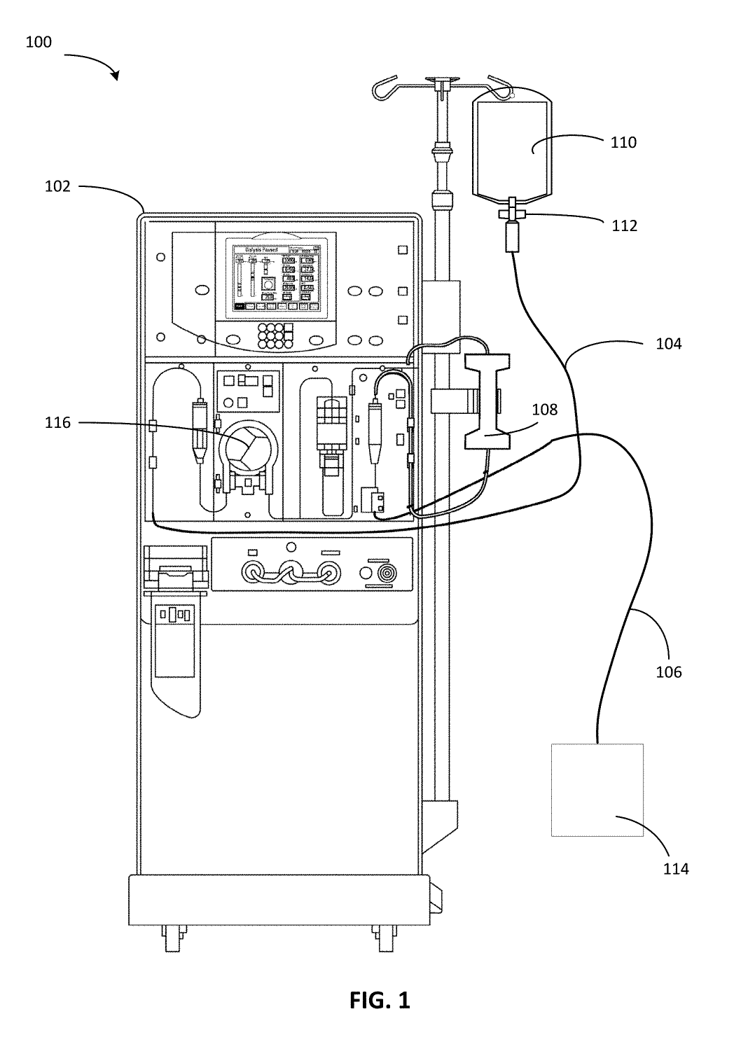

Referring to FIG. 1, a hemodialysis system 100 includes a hemodialysis machine 102 to which a blood line set including an arterial line 104 and a venous line 106 is connected. Before connecting the arterial line 104 and the venous line 106 to a patient, the hemodialysis machine is typically primed with a priming solution, such as saline, to remove air from a dialyzer 108 as well as other components of the blood line set. FIG. 1 shows the hemodialysis system 100 in a priming configuration in which the arterial line 104 is connected to a saline bag 110 via a spike port 112. The venous line 106 is connected to a waste container or drain 114. To carry out the priming procedure, a blood pump 116 is operated in a manner to force saline from the saline bag 110 through the arterial line 104, the blood pump 116, the dialyzer 108, and the venous line 106. The blood pump 116 is typically run for a set period or until a set volume of the saline has been pumped through the blood line set.

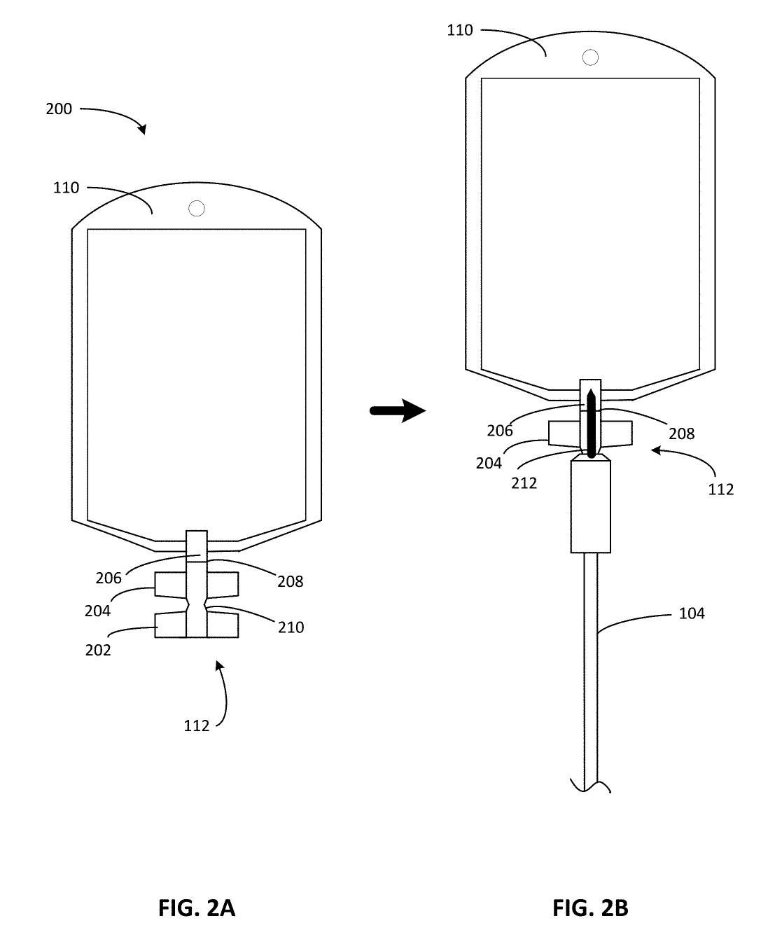

Referring to FIG. 2A, a saline bag 110 has a spike port 112 closing the saline bag. The spike port 112 has a first portion 202 and a second portion 204. The second portion 204 has a tip 206 which extends into an interior chamber of the saline bag 110. The tip 206 and the second portion 204 of the spike port 112 are separated by a membrane 208. To connect the spike port 112 to the body of the saline bag, the tip 206 of the spike port 112 is inserted into the saline bag 110 and then the saline bag assembly 200 is sterilized using an appropriate sterilization technique (e.g., heat sterilization). For example, during the heat sterilization process, a body of the saline bag 110 and the tip 206 are melted together and form a seal. The spike port 112 is manufactured such that there is substantially no knit line on the exterior of the tip 206, which allows the saline bag 110 and the spike port 112 to form a non-leaking, sterile seal during heat sterilization. Traditionally, a solvent (e.g. cyclohexane) would be required to attach and seal a port to a saline bag. However, the smoother outer surface of the tip 206, allows this expensive and toxic step to be removed.

To prepare to open the saline bag 110, the first portion 202 of the spike port 112 is twisted off and removed. The first portion 202 and the second portion 204 are separated by a wall 210 which is designed to tear at a set height defined by a thinned portion of the wall. Removing the first portion 202 from the second portion 204 exposes the membrane 208 between the tip 206 and the second portion 204. Referring to FIG. 2B, a spike 212 is inserted through the membrane 208 to access saline solution inside the saline bag 110. The spike 212 may be an ISO-compliant spike or a non-ISO compliant spike as the spike port 112 is formed from a flexible material and can interface with either spike type. The saline solution travels through the spike 212 into the arterial line 104 for priming of the blood line set before hemodialysis, as shown in FIG. 1.

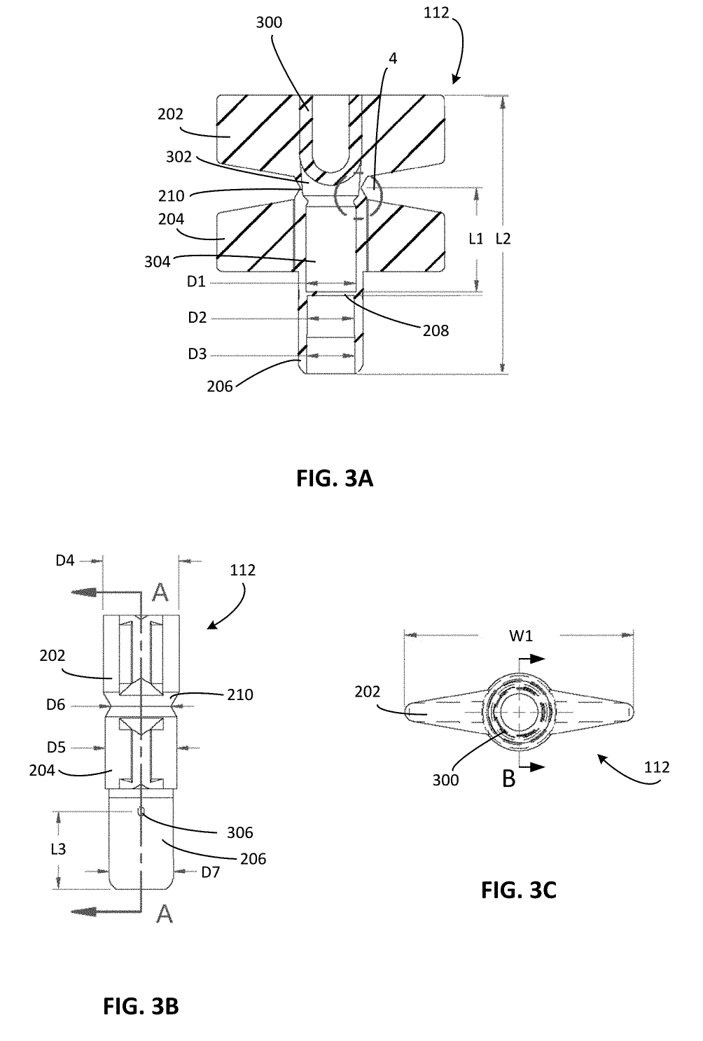

Referring to FIG. 3A, a plug 300 is positioned in a first chamber 302 of the first portion 202 of the spike port 112. The plug 300 is interference fit with a wall that defines the first chamber 302 such that the plug 300 seals the first chamber 302. The plug 300 can seal the first chamber 302 without the use of welding, making the manufacturing process faster and less expensive. The plug 300 also provides structural stability to the first portion 202 of the spike port 112. This increased structural stability allows removal of the first portion 202 from the second portion 204 with minimal deformation of the wall 210 and the second portion 204 into which a spike 212 will be inserted. Deformation of the wall 210 and/or the second portion 204, especially a second chamber 304 in the second portion 204, can increase the insertion force required to insert the spike 212 into the saline bag 110.

The second chamber 304 in the second portion 204 of the spike port 112 extends, along a length L1, which may be approximately 11.0 mm to 11.4 mm (e.g., approximately 11.2 mm), from the wall 210 separating the first portion 202 and the second portion 204 to the membrane 208. The second chamber has a diameter D1, which may be approximately 5.2 mm to 5.6 mm (e.g., approximately 5.4 mm). The tip 206 of the spike port 112 has a diameter D2 adjacent to the membrane 208 and a diameter D3 at the end of the tip 206. D2 may be approximately 4.9 mm to 5.3 mm (e.g., approximately 5.1 mm), and D3 may be approximately 5.0 mm to 5.4 mm (e.g., approximately 5.2 mm). The membrane 208 may have a thickness of approximately 0.3 mm to 0.5 mm (e.g., approximately 0.4 mm). The spike port 112 has a length L2, which may be approximately 29.5 mm to 30.5 mm (e.g., approximately 30 mm).

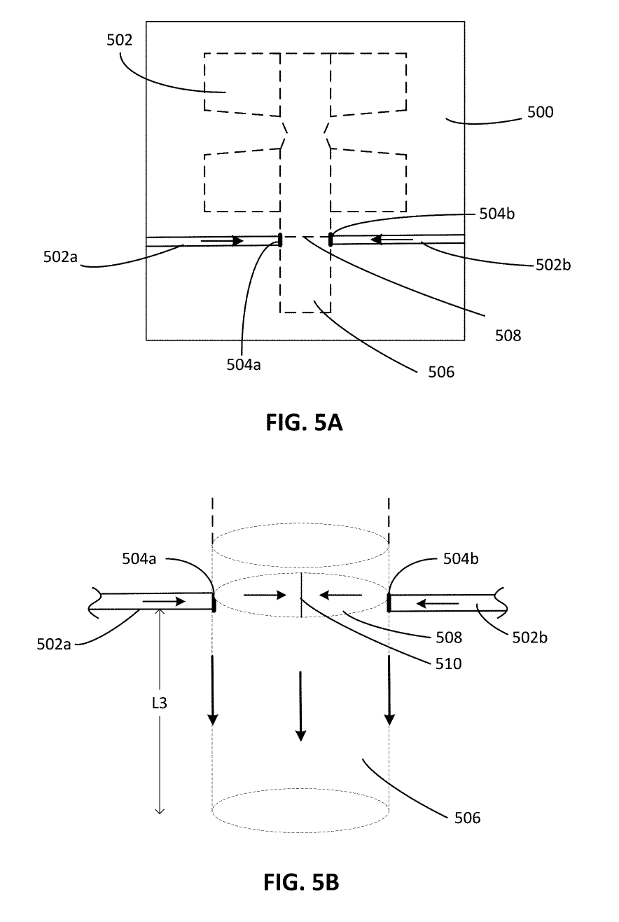

Turning to FIG. 3B, the first portion 202 has an exterior diameter D4 and the second portion 204 has an exterior diameter D5. D4 may be approximately 8.1 mm to 8.5 mm (e.g., approximately 8.3 mm) and D5 may be approximately 7.6 mm to 8.0 mm (e.g., approximately 7.8 mm). The wall 210, at its narrowest circumference, has a diameter D6, which may be approximately 6.3 mm to 6.7 mm (e.g., approximately 6.5 mm). The tip 206 of the spike port 112 has an exterior diameter D7, which may be approximately 6.8 mm to 7.2 mm (e.g., approximately 7.0 mm). A first injection mark 306 is visible on the outside of the tip 206 of the spike port 112 and corresponds to a position of an injection gate during the injection molding manufacturing process for making the spike port 112. The first injection mark 306 is positioned along axis A and at a distance L3 from the end of the spike port 112. L3 may be approximately 8.3 mm to 8.7 mm (e.g., approximately 8.5 mm). There is a second injection mark, also positioned along axis A and at a distance L3 from the end of the spike port 112, on the opposite side of the tip 206 of the spike port 112 from the first injection mark 306. The injection molding process will be discussed below in relation to FIG. 5.

Referring to FIG. 3C, the spike port 112 may have a width W1, which may be approximately 24.0 mm to 25.0 mm (e.g., approximately 24.5 mm).

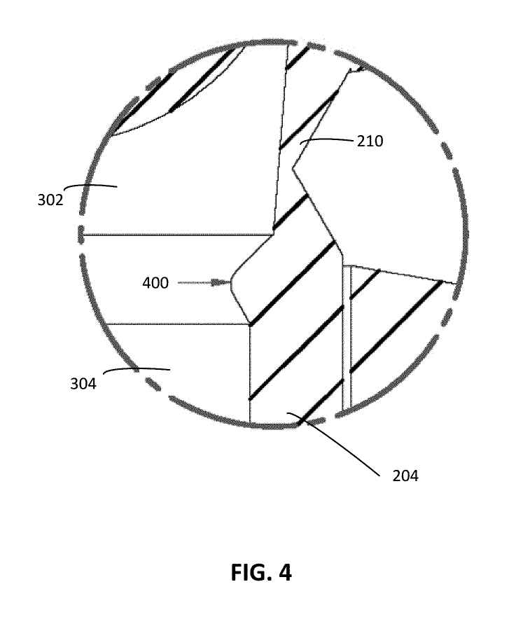

Turning to FIG. 4, a sealing ring 400 extends from the second portion 204 of the spike port 112 into the second chamber 304. Upon removal of the first portion 202 of the spike port 112, and spiking the membrane 208 with a spike 212, the sealing ring 400 surrounds and interfaces with the spike 212 to seal the spike 212 to the spike port 112. The sealing ring 400 has an interior diameter, which may be approximately 4.8 mm to 5.0 mm (e.g., approximately 4.9 mm). The inner diameter of the sealing ring 400 is about 0.0 mm to 0.5 mm smaller than the outer diameter of an ISO-compliant spike. The flexibility of the sealing ring 400 and the second portion 204 of the spike port 112 allows the spike port 112 to be used with an ISO-compliant spike or a non-ISO compliant spike. The sealing ring 400 can interface and form a seal with either spike type.

Turning to FIG. 5A, a mold 500 is used to form a spike port 112 during an injection molding process. During the injection molding process, a plasticized polyvinyl chloride (PVC) material is injected into the mold 500. The positioning of injection gates 504a and 504b in the mold 500 directs the flow of the PVC material into different portions of the mold 500 at different times. The PVC material flows through runners 502a and 502b before reaching the injection gates 504a and 504b.

Turning to FIG. 5B, the injection gates 504a and 504b are positioned in the mold such that, when forming the membrane 208 and the tip 206 of the spike port 112, a membrane zone 508 of the mold 500 is filled with PVC material before a tip zone 506 of the mold. In forming the membrane 208 in the membrane zone 508 of the mold 500, PVC material flows through the injection gates 504a and 504b and into the membrane zone 508. When the PVC material from injection gate 504a meets the PVC material from injection gate 504b, a knit line 510 is produced. The knit line 510 corresponds approximately with axis B shown in FIG. 3C.

After forming the membrane 208 in membrane zone 508, the PVC material flows into the tip zone 506 of the mold 500. As the PVC material from injection gate 504a has already met the PVC material from injection gate 504b in the membrane zone, substantially no knit line is created on the exterior of the tip 206. Because substantially no knit line is created on the exterior of the tip 206, the tip has a surface roughness (Rₐ) of 0.56 μm, which allows the tip 206 to be sealed to a body of a saline bag during a heat sterilization process without the use of a solvent.

At the end of the injection cycle, the tip 206 is moved to compress the PVC material in the membrane zone 508 of the mold 500 to the required membrane thickness, which may be approximately 0.3 mm to 0.5 mm (e.g., approximately 0.4 mm). The motion of the tip 206 to compress the membrane 208 to the required membrane thickness can be controlled by a hydraulic cylinder with magnetic sensors.

While the spike ports of the embodiments shown and discussed above include spike ports connected to a saline bag for use in priming a hemodialysis machine, spike ports may be used on bags for other types of fluids or uses. For example, a spike port may be used to close a bag of dialysate or other medical solutions. In another example, a spike port may be used with a bag containing a medication, or a bag of another medical fluid, which may be directly administered to a patient. For example, a saline bag with a spike port may be directly connected to a patient through an IV for rehydration.

While the spike ports of the embodiments shown and discussed above appear as the only port on a saline bag, a saline bag may have one or more additional ports. For example, a spike port may be connected to a saline bag with an additional infusion port which allows for the infusion of medications, vitamins, or other additives into the saline bag. A spike port may be used to access the infused solution inside of the bag.

While certain embodiments describe a medical solution bag used with a hemodialysis system, the medical solution bags and spike ports described herein can be used with other types of blood treatment systems, including peritoneal dialysis systems, hemofiltration systems, hemodiafiltration systems, apheresis systems, etc. Additionally, it should be understood that the medical solution bags and spike ports described herein can be used with any of various other medical treatment systems that do not relate to blood treatments.

While the spike ports of the embodiments shown and discussed above are described as being formed from PVC plasticized with DEHP or DEHP free, other materials may be substituted. For example, a spike port may be formed from another plastic material, including polyethylene, polypropylene, and poly (ethylene-vinyl acetate) (PEVA).

While the spike ports of the embodiments shown and discussed above are described as not requiring a solvent (e.g. cyclohexane) for assembly and sealing with a saline bag, a solvent may nonetheless be used to create and/or reinforce the seal.

While the spike ports discussed above have been described as having a thinned wall portion to allow the first portion to be removed from the second portion, other techniques can be used to enable the removal of the first portion from the second portion. For example, a spike port may have a pull off tab or pull off cover which may enable removing the first portion from the second portion.

While the spike ports discussed above have a knit line in the membrane, a knit line may be located in another area of the spike port that would not interfere with assembly and sealing the spike port to a saline bag. For example, the location of the injection mold gates could be arranged so a knit line would form on the first portion of the spike port, which is removed before spiking.

1. A molded spike port comprising:
a first portion defining a first chamber;
a plug disposed in the first chamber and configured to form a seal with the first portion;
a second portion defining a second chamber, the second portion comprising a sealing ring configured to seal the spike port to a spike;
a tip configured to be inserted into a fluid bag, the tip comprising a membrane; and
a wall between the first portion and the second portion, wherein the wall is configured to break when the first portion is twisted relative to the second portion.

2. The molded spike port of claim 1, wherein the membrane has a thickness of 0.3 mm to 0.5 mm.

3. The molded spike port of claim 1, wherein the sealing ring is configured to seal spikes of multiple configurations.

4. The molded spike port of claim 1, wherein one of the configurations is ISO-compliant and another of the configurations is non-ISO compliant.

5. The molded spike port of claim 1, wherein the sealing ring has an inner diameter of 4.8 mm to 5.0 mm.

6. The molded spike port of claim 1, wherein the tip of the spike port comprises a smooth outer surface configured to seal the tip of the spike port to the fluid bag via heat sterilization.

7. The molded spike port of claim 6, wherein the smooth outer surface has a surface roughness of 0.56 μm.

8. The molded spike port of claim 1, wherein the plug is interference fit with the first chamber.

9. The molded spike port of claim 1, wherein the wall comprises a thinned portion with a wall thickness that is thinner than a remainder of the wall.

10. The molded spike port of claim 1, wherein the spike port is made from plasticized polyvinyl chloride.

11. A method of manufacturing a molded spike port, the method comprising:
injection molding a spike port, comprising:
flowing a material through two injection mold gates on opposing sides of a tip zone of a spike port mold, the injection mold gates being positioned a predetermined length from an end of the tip of the mold;
filling a membrane zone of a mold such that material from the two injection mold gates meets in the membrane zone;
filling the tip zone of the spike port mold with the material; and
compressing the material in the membrane zone to form a membrane with a predetermined membrane thickness.

12. The method of claim 11, wherein the predetermined membrane thickness is 0.3 mm to 0.5 mm.

13. The method of claim 11, wherein the injection mold gates are positioned approximately 8.3 mm to 8.7 mm from the end of the tip zone of the mold.

14. The method of claim 11, wherein a knit line is formed in the membrane zone when the material from the two injection mold gates meets in the membrane zone.

15. The method of claim 11, wherein filling the tip zone of the spike port mold creates a spike port tip with a smooth outer surface.

16. The method of claim 15, wherein the smooth outer surface has a surface roughness of 0.56 μm.

17. The method of claim 11, further comprising filling a first portion zone of the spike port mold and a second portion zone of the spike port mold such that the first portion and the second portion are connected at a thinned portion.

18. The method of claim 11, further comprising filling a first portion zone of the spike port mold and a second portion zone of the spike port mold such that the second portion and the tip are connected at a distance further from the end of the tip zone of the mold than the two injection mold gates.

19. The method of claim 11, wherein a hydraulic cylinder compresses the material in the membrane zone to the predetermined thickness.
